(19) United States
(12) Patent Application Publication
(10) Pub. No.: US 2013/0157259 A1
(43) Pub. Date: Jun. 20, 2013

(54) METHOD OF AMPLIFYING DNA FROM RNA IN SAMPLE AND USE THEREOF

(71) Applicant: SAMSUNG ELECTRONICS CO., LTD., Suwon-si (KR)

(72) Inventors: Ko-bong CHOI, Osan-si (KR); Joo-won RHEE, Yongin-si (KR); Sea-hee KIM, Seoul (KR)

(73) Assignee: SAMSUNG ELECTRONICS CO., LTD., Suwon-si (KR)

(21) Appl. No.: 13/715,955

(22) Filed: Dec. 14, 2012

(30) Foreign Application Priority Data
Dec. 15, 2011 (KR) 10-2011-0135775
Nov. 16, 2012 (KR) 10-2012-0130514

(51) Int. Cl. C12Q 1/68; C12P 19/34

(52) U.S. Cl. CPC C12Q 1/686; C12P 19/34
USPC 435/5; 435/91.21; 435/6.12; 435/6.11; 435/194

(57) ABSTRACT

Provided are methods of efficiently amplifying DNA from RNA in sample, methods of efficiently estimating an amount of RNA in a sample, and compositions for efficiently amplifying DNA from RNA in a sample.

### CROSS-REFERENCE TO RELATED APPLICATIONS

This application claims the benefit of Korean Patent Application No. 10-2011-0135775, filed on Dec. 15, 2011, and 10-2012-0130514, filed on Nov. 16, 2012, in the Korean Intellectual Property Office, the disclosure of which is incorporated herein in its entirety by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,308 Byte ASCII (Text) file named “711818_ST25.txt,” created on Dec. 14, 2012.

### BACKGROUND

1. Field

The present disclosure relates to methods of efficiently amplifying DNA from RNA in a sample, methods of efficiently estimating an amount of RNA in a sample, and compositions for efficiently amplifying DNA from RNA in a sample.

2. Description of the Related Art

Amplifying small quantities of RNA obtained from limited biological samples is a fundamental, critical process in gene assay procedures, including quantitative assays, such as gene-expression assays of RNA, and qualitative assays, such as sequence analysis of RNA. RNA assays have many applications, for example, in developing a drug target for the development of new drug, in developing a diagnosis marker, in identifying gene mutations, or in diagnosing a disease generated by a gene mutation, such as a genetic disease, or a cancer.

Generating or amplifying DNA from RNA involves first synthesizing cDNA from an RNA template in a reaction catalyzed by reverse transcriptase and DNA polymerase and then synthesizing DNA from the cDNA. Reverse transcription is performed by binding a primer (often, but not always, an oligo-dT primer) to an RNA template and extending the primer to generate a cDNA.

However, the rate at which RNA is transcribed to cDNA is affected by primer binding, RNA sequence, and RNA 3-dimensional structure. As such an amplification bias may occur wherein some RNA is easily synthesized into DNA and other RNA is less easily synthesized into DNA. That is, when a plurality of RNA is present in a sample, some RNA may be preferentially transcribed into DNA.

In addition, when DNA is synthesized from cDNA, a DNA dependent DNA polymerization reaction, such as PCR or multiple displacement amplification (MDA), may be used. Even in this process, due to a various single-stranded cDNA structures and sequences, primer binding efficiency may vary according to a target, thereby leading to amplification bias.

Typically, when DNA is amplified from RNA, generation or amplification may occur with bias.

### SUMMARY

Provided are methods of efficiently amplifying DNA from RNA in sample. In one aspect, provided herein is a method of amplifying DNA from RNA in a sample, the method comprising: incubating a sample having one or more RNA in the presence of RNA ligase, which ligates the 5′ terminal and 3′ terminal of the RNA to form circular RNA; combining the circular RNA with a primer hybridizable to a region of the circular RNA or a sequence complementary to the circular RNA, RNA-dependent DNA polymerase, and DNA-dependent DNA polymerase to form an aqueous component; introducing the aqueous component into a microcompartment of a water-in-oil emulsion; and amplifying DNA from the circular RNA. Also provided is a method for efficiently estimating the amount of RNA in a sample, and a composition for efficiently amplifying DNA from RNA in a sample.

### BRIEF DESCRIPTION OF THE DRAWINGS

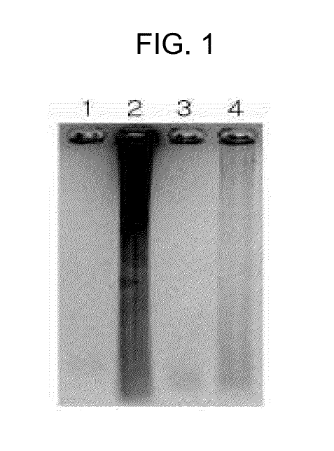

These and/or other aspects will become apparent and more readily appreciated from the following description of the embodiments, taken in conjunction with FIG. 1, which is an image showing electrophoresis results of RNA amplification.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

A method of amplifying DNA from RNA in a sample, according to an embodiment of the present invention, includes: incubating a sample including one or more RNA in the presence of RNA ligase, which ligates the 5′ terminal and the 3′ terminal of the RNA to form circular RNA; combining the circular RNA with a primer hybridizable to a region of the circular RNA or a sequence complementary to the circular RNA, RNA-dependent DNA polymerase, and DNA-dependent DNA polymerase to form an aqueous component; introducing the aqueous component into a microcompartment of a water-in-oil emulsion; and amplifying DNA from the circular RNA.

The method includes incubating a sample including one or more RNA in the presence of RNA ligase, which ligates the 5′ terminal and the 3′ terminal of the RNA to form circular RNA.

The ligation may be a ligation in the same molecule. The RNA may be mRNA, tRNA, rRNA, or a combination thereof. The sample may include only RNA as a nucleic acid, or may be an extract in which RNA is concentrated. The sample may include RNA isolated from a biological sample by a RNA isolation process. The RNA may be synthetic, semi-synthetic, or a transcriptome expressed from cell or virus. The RNA may be stored RNA. The storing may be achieved by any known method. The storing may be performed for 1 year or more, for example, 1 year to 10 years. The RNA may be derived from a tissue that is stored at room temperature after having been frozen or formalin-fixed paraffin embedded. Methods for isolating RNA from a biological sample are well known in the art. For example, a trizol method may be used.

The sample may include a RNA decomposition product isolated from a biological sample. The sample may include RNA isolated from a formalin-fixed paraffin embedded (FFPE) tissue sample. Natural mRNA of eukaryotic cells may have a 5′-cap structure and a 3′-poly(adenylate) sequence. However, mRNA may decompose into a decomposition product during storage or treatment. In this regard, the decomposition product may not have a 5′-cap structure and 3′-poly(adenylate) sequence of the natural mRNA. According to an embodiment of the present invention, mRNA used in the amplification may include RNA that does not have the structure of natural mRNA. The sample may include RNA having a 5′-cap and a 3′-OH; RNA having a 5′-cap and a 3′-monophosphate; RNA having a 5′-OH and a 3′-monophosphate; RNA having a 5′-OH and a 3′-OH; RNA having a 5′-monophosphate and a 3′-OH; RNA having a 5′-monophosphate and a 3′-monophosphate; or a combination thereof.

The 5′-cap structure may include a structure in which 7-methylguanylate is linked to a 5′-OH of sugar of the 5′-terminal via triphosphate linkage. The 3′-OH and/or 2′-OH of the terminal guanylate of 5′-cap structure and 2′-OH of first and second nucleotides from the 5′-terminal may be methylated.

The RNA may have a 5′-phosphorylation and a 3′-OH or may be modified to have a 5′-phosphorylation and a 3′-OH. The RNA may have a length of about 10 to about 100 nt, for example, about 19 to about 40 nt.

The RNA ligase may catalyze a molecular ligation. The RNA ligase may be, for example, T4 RNA ligase 1, T4 RNA ligase 2, Circligase™ (a thermostable ATP-dependent ligase that catalyzes intramolecular ligation (i.e. circularization) of ssDNA templates having a 5′-phosphate and a 3′-hydroxyl group), or a combination thereof. Circligase™ is found to capable of catalyzing intramolecular ligation of ssRNA template having a t′-phophate and a 3′-hydroxyl group by the inventors.

The incubation may be performed under conditions appropriate for the activity of the RNA ligase. The incubation may be performed in the presence of elements that are required for the activity of the RNA ligase.

The method may include the introducing of an aqueous component including one or more circular RNA, a primer hybridizable to a region of the circular RNA or a sequence complementary to the circular RNA, RNA-dependent DNA polymerase, and DNA dependent DNA polymerase into a microcompartment of water-in-oil emulsion.

The introducing of an aqueous component into a microcompartment present in water-in-oil emulsion is known in the art, the microcompartment being the aqueous droplets of the emulsion (the terms “microcompartment” and “droplet” or “aqueous droplet” are used interchangeably herein). For example, an aqueous component may be mixed with an oily component to form a water-in-oil emulsion, including a microcompartment containing at least a part of the aqueous component. The microcompartment may have a mean diameter of 10 μm or less. For example, the microcompartment may have a mean diameter of about 100 nm to about 10 μm, about 100 nm to about 5 μm, about 100 nm to about 3 μm, or about 100 nm to about 2 μm.

The oily component refers to a lipophilic component that is not mixable with water. The oily component may include mineral oil, for example, silicon oil.

The emulsion including the microcompartment may further include, in addition to oily component, a surfactant. The surfactant may stabilize a state of microcompartment in emulsion or a state of emulsion. The surfactant may increase thermal stability of emulsion. The surfactant may also be called an emulsifier. The surfactant may include one or more of yolk, lecithin, sodium stearoyl lactylate, emulsifying wax, polysorbate 20, and cetereth 20. The surfactant may be a non-ionic surfactant. The non-ionic surfactant may be a non-ionic surfactant that has a hydrophilic-lipophilic balance (HLB) value of 4 or less. The HLB value may be calculated based on Griffin's equation below:

HLB value=20×MH/M (MH: molecular weight of hydrophilic moiety, and M: molecular weight of surfactant)

The non-ionic surfactant may include one or more of Span 80 (sorbitan monooleate: Fluka, Japan), Tween 80 (polyoxyethylene sorbitan monooleate, Nakarai, Japan), Triton X-100 (t-octylphenoxypolyethoxyethanol), Sun soft No. 818SK (condensed polyglycerinester ricinoleate: Sun chemical, Japan), and Sun soft O-30V (glycerin oleate: Sun chemical, Japan).

The circular RNA may be a dilution in which 3 molecules or less, for example, 2 molecules or 1 molecule or less is included in a microcompartment.

The primer may be a sequence specific primer or a primer having a random sequence. The primer may have a length of about 10 to about 100 nt, for example, about 10 to about 50 nt, about 10 to about 40 nt, about 10 to about 30 nt, or about 15 to about 30 nt. The random primer have a length of about 5 to about 10 nt, for example, about 5 nt, about 6 nt, about 7 nt, about 8 nt, about 9 nt, or about 10 nt. The primer may be a single-stranded DNA. The primer may include only reverse primer R that is complementary to the RNA. According to another embodiment of the present invention, the primer may include at least one selected from reverse primer R that is complementary to the RNA and forward primer F that is complementary to a sequence complementary to the RNA.

The RNA-dependent DNA polymerase may include an enzyme having RNA-dependent DNA polymerization activity. The RNA-dependent DNA polymerase may be used exchangeably with reverse transcriptase. The RNA-dependent DNA polymerase may be HIV-1 reverse transcriptase derived from human immunodeficiency virus type 1, M-MLV reverse transcriptase derived from moloney murine leukemia virus, AMV reverse transcriptase derived from avian myeloblastosis virus, HIV reverse transcriptase, or a combination thereof.

The DNA-dependent DNA polymerase may include an enzyme having RNA-dependent DNA polymerization activity. The DNA-dependent DNA polymerase may have a strand displacement activity. The DNA-dependent DNA polymerase may be selected from the group consisting of Bst DNA polymerase, exonuclease minus, pyrophage 3173 polymerase, Tth polymerase, and a combination thereof. For example, the DNA polymerase may be Bst DNA polymerase, exonuclease minus. The Bst DNA polymerase, exonuclease minus are each a 67 kDa Bacillus stearothermophilus DNA polymerase protein (large fragment) that has 5′ to 3′ polymerase activity and strand displacement activity and does not have 3′ to 5′ exonuclease activity. In addition, it has reverse transcription activity. Bst DNA polymerase, exonuclease minus may be used in nucleic acid amplification, whole genome amplification, multiple displacement amplification, and the like, including isothermal amplification. M-MLV, AMV, and HIV reverse transcriptases include reverse transcription activity, ribonuclease activity, and

DNA-dependent DNA polymerase activity. Bst DNA polymerase, exonuclease minus is known to have higher DNA-dependent DNA polymerization activity compared to RNA-dependent DNA polymerization activity. Thus, when a sample containing RNA only is firstly incubated with the Bst DNA polymerase, exonuclease minus, DNA is synthesized from the RNA by a reverse transcription reaction catalyzed by the RNA-dependent DNA polymerization activity of the Bst DNA polymerase, exonuclease minus, and then once a DNA, for example, a single-stranded DNA is formed, DNA is synthesized from synthesized DNA by a reaction catalyzed by the DNA dependent DNA polymerization activity of the Bst DNA polymerase, exonuclease minus. Accordingly, the primer may include only a primer that is complementary to RNA template, or may additionally include a primer that is complementary to the formed single-stranded DNA.

The aqueous component may further include a reagent for reverse transcription reaction or DNA polymerization reaction. The reagent for reverse transcription reaction or DNA polymerization reaction may further include a reagent for RNA-dependent DNA polymerization reaction, DNA-dependent DNA polymerization reaction, and a combination thereof. The reagent may further include reaction buffer, ribonucleotide triphosphate, deoxyribonucleotide triphosphate, or a coenzyme or cofactor that is required for the activity of polymerase.

The RNA may exist as, on average, 3 molecules or less, for example, 2 molecules or less, or 1 molecule or less per microcompartment.

According to an embodiment of the present invention, the introducing may include: preparing an aqueous solution including one or more circular RNA, a primer hybridizable to a region of the circular RNA or a sequence complementary to the circular RNA, a RNA-dependent DNA polymerase, and a DNA-dependent DNA polymerase; and mixing the aqueous component, an oily component, and a non-ionic surfactant to prepare a water-in-oil emulsion.

The preparing may include mixing the circular RNA, the primer, RNA-dependent DNA polymerase, and DNA-dependent DNA polymerase in an aqueous medium, for example, water, PBS, or a buffer. The buffer may be a DNA polymerase reaction buffer or a PCR reaction buffer. The RNA-dependent DNA polymerase and the DNA-dependent DNA polymerase may each be used in such an amount that, on average, 1 or more molecules are included in each formed microcompartment.

The preparing of water-in-oil emulsion may be performed by mixing the aqueous component, the oily component, and the non-ionic surfactant. The mixing may be performed with or without stirring. In addition, the mixing may be performed by applying an ultrasound wave. The aqueous component and the oily component are described above. The non-ionic surfactant may be a non-ionic surfactant that has a HLB value of 4 or less. HLB may be calculated based on the Griffin's equation. The non-ionic surfactant may include one or more of Span 80 (sorbitan monooleate: Fluka, Japan), Tween 80 (polyoxyethylene sorbitan monooleate, Nakarai, Japan), Triton X-100 (t-octylphenoxypolyethoxyethanol), Sun soft No. 818SK (condensed polyglycerinester ricinoleate: Sun chemical, Japan), and Sun soft O-30V (glycerin oleate: Sun chemical, Japan).

According to an embodiment of the present invention, the amplifying may be performed under suitable conditions for at least one of RNA-dependent DNA polymerization and DNA-dependent DNA polymerization. The amplifying may be performed in an emulsion including the microcompartment. The amplifying may be performed for a sufficient period of time so that DNA amplification in each microcompartment reaches a saturation state. The time for the amplifying may be 1 hour or more, for example, about 1 hour to about 10 hours, about 1 hour to about 5 hours, about 2 hours to about 5 hours, about 1 hour to about 3 hours, or about 1.5 hours to about 2.5 hours. The term “amplifying” may include generation or amplification of DNA. The generated product may be a multimeric nucleic acid having a region in which the same sequence is repeated.

A temperature for the amplifying may be not limited as long as at least one of RNA-dependent DNA synthesis and DNA-dependent DNA synthesis occurs. The amplifying may be performed at the temperature of about 40° C. to about 50° C. The amplifying may be performed in isothermal conditions. The amplifying may be performed in an isothermal condition of about 40° C. to about 50° C. The term “isothermal condition” refers to a condition in which thermal cycling does not happen. In addition, the amplifying may be performed in thermal cycling conditions.

The amplifying may be performed by strand displacement amplification (SDA), for example, rolling circle amplification. The strand displacement amplification, for example, rolling circle amplification, may indicate a unidirectional nucleic acid amplification process for quickly synthesizing multiple copies of an original molecule of DNA or RNA, such as plasmid, genome of bacterial phage, or viroid circular RNA genome. Some eukaryotic viruses amplify DNA based on rolling circle mechanism. The rolling circle amplification includes attaching a random or specific primer to a single-stranded original nucleic acid, and template-dependently attaching a nucleotide to a 3′-OH terminal of the primer to extend a sequence. When the extended sequence meets a double-stranded region, such as a region to which the primer is bound, due to DNA strand displacement activity of DNA polymerase, a single-stranded DNA may extend from the template by displacement. The primer may be a region having a 3′-OH that is generated by cleavage, for example, by nicking of a double-stranded nucleic acid.

A method of estimating an amount of RNA in a sample, according to an embodiment of the present invention, includes: incubating a sample including one or more RNA in the presence of RNA ligase, which ligates the 5′ terminal and the 3′ terminal of RNA to form circular RNA; combining the circular RNA with a primer hybridizable to a region of the circular RNA or a sequence complementary to the circular RNA, RNA-dependent DNA polymerase, and DNA-dependent DNA polymerase to form an aqueous component; introducing the aqueous component into a microcompartment of a water-in-oil emulsion; amplifying DNA from the circular RNA; and estimating the amount of a RNA species in the sample from the amount of an amplified DNA species.

The incubating for the generation of circular RNA, the introducing the aqueous component into microcompartment of water-in-oil emulsion, and the amplifying DNA from the circular RNA are described above.

The method includes estimating an amount of a RNA species in the sample from the amount of an amplified DNA species. A method of measuring the amount of an amplified DNA species is known in the art. For example, each DNA may be isolated by electrophoresis and a concentration thereof may be measured. As another example, a quantity of a target DNA may be measured by quantitative PCR. The amplified DNA species may have a correlation with a RNA species in terms of quantity and ratio. That is, since, from among multiple RNA species, a smaller number of molecules are introduced into a single microcompartment and amplified, the quantity and ratio of different RNA species may be measured with less bias, or without bias. This bias may further decrease because the amplification reaction proceeds until it reaches a saturation state. That is, DNA may be sufficiently amplified from RNA or DNA included in each microcompartment, thereby enabling the reaction to continue until space and enzymatic action are restricted to the point that no further amplification occurs in each microcompartment. In other words, a preference exhibited by a polymerase towards one type of RNA over another type of RNA is reduced or eliminated by separating the RNA into their own individual microcompartments, and allowing amplification to proceed to its saturation point. The relative amount of one species of RNA as compared to other species of RNA is represented, then, in the amount of amplification of one type of RNA compared to the amount of amplification of another type of RNA.

The method may further include estimating an amount ratio of different kinds of RNA species in the sample from an amount ratio of different kinds of amplified DNA species.

A composition for amplifying DNA from RNA in a sample, according to an embodiment of the present invention, includes a microcompartment of a water-in-oil emulsion including an aqueous component including the circular RNA, a primer that is hybridizable to a region of the circular RNA or a sequence complementary to circular RNA, RNA-dependent DNA polymerase, and DNA-dependent DNA polymerase.

The microcompartment and components that constitute the microcompartment are described above. The composition may be an emulsion including a microcompartment.

The method of amplifying DNA from RNA in a sample, according to an embodiment of the present invention, may enable DNA to be generated or amplified from RNA with less bias.

The method of estimating an amount of RNA in a sample, according to an embodiment of the present invention, may enable the quantity of RNA in a sample to be efficiently estimated.

The composition for the amplification of DNA from RNA in a sample, according to an embodiment of the present invention, may enable DNA to be efficiently generated or amplified from RNA in a sample.

Hereinafter, one or more embodiments of the present invention are described in detail with reference to Examples. However, Examples are presented herein for illustrative purpose only, and do not limit the one or more embodiments of the present invention.

### EXAMPLE 1

(1) RNA Preparation

(1.1) Preparation of a Linear Double-Stranded DNA Fragment

To manufacture three kinds of double-stranded DNA fragments, universal human reference RNA (Stratagene, CAT: #74000) was used as a template to perform RT-PCR.

In detail, 1 pg of UHR RNA, 1 μl of 2 pmole target specific primer (reverse), 1 μl of 10 mM dNTP mix, and 13 μl of a total volume of H₂O were added to 20 μl of a reaction vial, and then, the mixture was incubated at the temperature of 65° C. for 5 minutes, and then cooled in ice for 1 minute. Thereafter, 4 μl 5× first strand buffer (Invitrogen, Superscript™ III buffer), 1 μl of 0.1 M DTT, 1 μl of 40 U/μl RNaseOUT (Invitrogen), and 1 μl of 200 U/μl SuperScript™ III reverse transcriptase (Invitrogen) were added thereto, and then, the resultant mixture was incubated at the temperature of 45° C. for 30 minutes. SuperScript™ III reverse transcriptase is a manipulated version of M-MLV RT having reduced RNaseH activity and increased thermal stability. This enzyme may be used at the temperature of 55° C. or lower to synthesize a first strand cDNA.

Subsequently, a reaction mixture in a total volume of 20 μl was prepared including 10 μl of 2× HS Prime Taq premix (GeNet BIO), 0.5 μM forward primer, 0.5 μM reverse primer, 2 μl of reverse transcriptase reaction product, and 6 μl of water. The reaction mixture was incubated at the temperature of 94° C. for 10 minutes, and then a thermal cycling of 30 seconds at the temperature of 94° C., 30 seconds at the temperature of 55° C., and 30 seconds at the temperature of 72° C. was repeatedly performed 30 times, and then, the result was incubated at the temperature of 72° C. for 5 minutes. HS Prime Taq premix includes a HS Prime Taq DNA polymerase, a reaction buffer, dNTP mixture, and a protein stabilizer. The HS Prime Taq DNA polymerase is designed for hot-start PCR. This enzyme does not have an activity at room temperature, and a functional activity thereof is recovered during incubation at the temperature of 94° C. for 10 minutes.

The linear double-stranded DNA fragment obtained from the incubation product was identified by electrophoresis. As a result, 128 bp Actin, 128 bp GUSB, and 125 bp TFRC DNA fragments were identified. The DNA fragments show that T7 promoter site is included in a 5′ terminal region.

(1.2) Preparation of RNA Fragment

In-vitro transcription was performed on the linear double-stranded DNA fragments obtained from (1.1) to produce mRNA. In detail, 50 μl of a reaction mixture including 100 ng of linear double-stranded DNA fragment obtained from (1.1), 2 μl of T7 RNA polymerase (Invitrogen), 1× reaction buffer (40 mM Tris-HCl, 6 mM MgCl₂, 10 mM DTT, 2 mM spermidine, pH 7.9 at the temperature of 25° C.), 7.5 mM NTP (ATP, CTP, GTP and UTP), and 25 μl of water was incubated at the temperature of 37° C. for 2 hours.

The obtained RNA was identified by electrophoresis. As a result, 106 bp Actin, 106 bp GUSB, and 103 bp TFRC RNA were identified. Deoxyoligonucleotide sequences used for RT-PCR are shown in Table1.

TABLE 1

| Name | SEQ ID NO: |
|---|---|
| T7_actin-100mer-F | 1 |
| T7_actin-100mer-R | 2 |
| T7_GUSB-100mer-F | 3 |
| T7_GUSB-100mer-R | 4 |
| T7_TFRC-100mer-F | 5 |
| T7_TFRC-100mer-R | 6 |
| Actin TaqMan probe | 7 |
| GUSB TaqMan probe | 8 |
| TFRC TaqMan probe | 9 |

(1.3) Self-Ligation of RNA

From the RNA product synthesized by the in-vitro transcription, RNA self-ligation was induced with 5′-pyrophosphohydrolase (RppH, New England Biolaboratories) and CircLigase™ II ssDNA Ligase (Epicentre) to produce circular RNA. CircLigase™ II ssDNA Ligase is a thermally stable enzyme that catalyzes intra-molecular ligation (that is, circularization) of ssDNA substrate having a 5′-monophosphate and a 3′-hydroxyl group. Linear ssDNA and ssRNA having about 15 or more bases may be circularized due to CircLigase™ II ssDNA Ligase.

In detail, 3 μg of the RNA product synthesized in (1.2), 10 μl NEB2 buffer (10×), and 10 μl RppH enzyme (5 U/μl) were mixed with water in an amount that makes a total volume of the resultant mixture to be 100 μl, and the mixture was incubated at the temperature of 37° C. for 1 hour.

From the reaction product, RppH treated RNA was obtained by purification with mirVana™ miRNA Isolation Kit (Ambion). 300 ng of RppH treated RNA, 2 μl of Circligase™ II buffer (10×), 1 μl of 50 mM MnCl₂, 4 μl of 5M Betaine, 1 μl of RNasin (40 unit/μl), and 1 μl of CircLigase™ II (100 U/μl) was mixed with water in an amount that makes a total volume of the resultant mixture to be 20 μl to obtain a reaction mixture. The reaction mixture was incubated at the temperature of 60° C. for 1 hour to obtain a circular RNA.

(2) Amplification from Circular RNA

A different quantity of circular RNA (Actin) was mixed with a solution of Tris-HCl (pH 8.0) 52.5 mM, KCl 70 mM, (NH₄)₂SO₄ 8.4 mM, MgCl₂ 14 mM, dNTP 1.4 mM, Tween 20 0.12%, random hexamer primer 26 μM, and Bst DNA polymerase 4.6 U/ul to prepare a reaction mixture with a total volume of 50 ul, and the reaction mixture was incubated. Amounts of RNA used herein were, respectively, 16 pg, 80 pg, 400 pg, 2 ng, and 10 ng. The incubation was performed by isothermal amplification at the temperature of 45° C. for 90 minutes. As a result, the obtained reaction product was subjected to electrophoresis to quantify amplified DNA.

TABLE 2

| RNA quantity | | | | | |
|---|---|---|---|---|---|
| used | 16 pg | 80 pg | 400 pg | 2 ng | 10 ng |
| Amplification | 103437 | 23375 | 4887 | 857 | 173 |
| fold | | | | | |
| CV(%) | 4.8 | 0.8 | 5.6 | 9.9 | 14 |
| Product | 1.6 | 1.8 | 1.9 | 1.7 | 1.7 |
| quantity (μg) | | | | | |
| Concentration | 33.1 | 37.4 | 39.1 | 34.3 | 34.7 |
| (ng/μl) | | | | | |

(3) Generation of Water-In-Oil Droplet

Span 80, tween 80, and Triton X-100 were added to 50 ml of mineral oil in such amounts that concentrations of Span 80, tween 80, and Triton X-100 were 4.5% (v/v), 0.4% (v/v), and 0.05% (v/v), respectively, to obtain an oil-surfactant mixture. Then, 400 ul of the oil-surfactant mixture was placed in a cryotube vial, and then, mixed by stirring with a 3x8 mm stir bar at a rotation rate of 1000 rpm for 5 minutes.

200 ul of aqueous phase including 20 mM Tris-HCl (pH 8.8, 25° C.), 10 mM (NH₄)₂SO₄, 10 mM KCl, 2 mM MgSO₄, 0.1% Triton X-100, forward primer 1 μM, reverse primer 1 μM (or 26 μM random hexamer), 1.3 mM dNTP, 4.8 unit/ul Bst DNA polymerase and 2 ng RNA synthesized in (1.3) was dropped into the cryotube vial containing the oil-surfactant mixture, and the resultant mixture was mixed by stirring at a rotation rate of 1000 rpm for 5 minutes.

As a result, an emulsion including an aqueous-phase containing droplet was obtained. The obtained droplet had a mean diameter of 3.33 μm (CV 40%).

(4) Polymerization Reaction

The emulsion was incubated at the temperature of 45° C. for 5 hours to cause a reverse transcription reaction and a DNA dependent DNA polymerization reaction. After the reactions, RNase was added thereto and incubation was performed thereon to decompose residual RNA.

In detail, 2 ng of circular RNA (Actin) was mixed with a solution of Tris-HCl (pH 8.0) 52.5 mM, KCl 70 mM, (NH₄)₂SO₄ 8.4 mM, MgCl₂ 14 mM, dNTP 1.4 mM, Tween 20 0.12% (v/v), forward primer 1 μM, reverse primer 1 μM (or 26 μM random hexamer), and Bst DNA polymerase 4.6 U/ul to prepare 200 ul of a total volume of a reaction aqueous phase, and then, a water-in-oil droplet was generated accordingly, and isothermal incubation was performed thereon at the temperature of 45° C. for 5 hours.

After the reaction was finished, the emulsion was centrifuged at a gravity of 13,000 g for 5 minutes to remove an oil phase therefrom. 1 ml of water-saturated diethyl ether was added to the aqueous phase to destroy the droplets of the emulsion to remove mineral oil. Electrophoresis was performed on the product obtained to confirm amplification.

FIG. 1 is an image showing electrophoresis results of RNA amplification. The reaction product was amplified to have branched structures, and various sizes were identified in an electrophoresis image. That is, the reaction product was amplified into multi-concatemer nucleic acid. In FIG. 1, lanes 1 and 2 show results obtained with an Actin gene specific primer, lanes 3 and 4 show amplification results obtained with random hexamer, lanes 1 and 3 show results obtained without RNA in a sample, and lanes 2 and 4 show results obtained with circular RNA (Actin) in a sample circular RNA (Actin).

### EXAMPLE 2

Three kinds of circular RNA were mixed at a ratio of Actin: 5 pg, TFRC: 50 fg, and GUSB: 5 fg, and then, the mixture was used as a sample and mixed with a solution of Tris-HCl (pH 8.0) 52.5 mM, KCl 70 mM, (NH₄)₂SO₄ 8.4 mM, MgCl₂ 14 mM, dNTP 1.4 mM, Tween 20 0.12%, 26 μM random hexamer, and Bst DNA polymerase 4.6 U/ul to prepare a total volume of 200 ul of a reaction aqueous phase, and a water-in-oil droplet was generated accordingly, and the result emulsion was subjected to isothermal incubation at the temperature of 45° C. for 15 hours (hereinafter referred to as “emulsion amplification)”.

With regard to the obtained product, quantitative RT-PCR (qRT-PCR) was performed in a PCR reaction solution including each of primers that were specific to 3 kinds of RNA. In detail, 0.1 μl of 100 μM forward primer, 0.1 μl of 100 μM reverse primer, 0.2 μl of 100 μM TaqMan™ probe, 10 μl of LIGHTCYCLER 480 PROBES MASTER (Roche), and 5 μl of the circular product were added to 100 μl tube, and then water was added thereto in an amount that makes the resultant mixture to be 20 μl, and then, qRT-PCR was performed with a Lightcycler 480 (Roche) device. Incubation was performed thereon while thermal cycling of 10 seconds at the temperature of 95° C. and 10 seconds at the temperature of 55° C. was repeatedly performed for 50 times.

With regard to initial RNA quantification, 1 μl of 2 pmole target specific primer (reverse direction), 1 μl of 10 mM dNTP mix, and 6 μl of water were added to 5 μl of 3 kinds of RNA mixtures in a tube, whereby the total volume of the mixture was 13 μl, and then, the resultant mixture was incubated at the temperature of 65° C. for 5 minutes, and then, cooled in ice for 1 minute. The contents of the tube was collected by a brief centrifugation. Thereafter, 4 μl of 5× first strand buffer (Invitrogen, Superscript™ III buffer), 1 μl of 0.1 M DTT, 1 μl of 40 U/μl RNaseOUT (Invitrogen), and 1 μl of 200 U/μl SuperScript™ III reverse transcriptase (Invitrogen) were mixed with the contents of the tube, and then, the resultant mixture was incubated at the temperature of 45° C. for 30 minutes to obtain cDNA product. cDNA product 5 μl of the obtained cDNA product was analyzed by, for example, qPCR of the amplification product.

When the respective RNA single molecules were amplified in a single emulsion, a competitive reaction was minimized, and thus, Pearson correlation of Ct values before and after the amplification, were almost the same as 0.99. Accordingly, it was confirmed that RNA molecules exist at the same ratio before and after the amplification.

Table 3 shows DNA amplification products obtained by qRT-PCR performed on a sample before and after emulsion amplification.

TABLE 3

| | Before | After |
|---|---|---|
| | amplification | amplification |
| | (Ct) | (Ct) |
| Actin | 25.99 | 23.60 |
| TFRC | 30.78 | 28.74 |
| GUSB | 33.06 | 30.21 |

| | |
|---|---|
| P. Correlation | 0.99 |

As shown in Table 3, it was confirmed that obtained 3 kinds of DNA amplification products exist at almost the same ratio as that of the initial RNA.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A method of amplifying DNA from RNA in a sample, the method comprising:
incubating a sample having one or more RNA in the presence of RNA ligase, which ligates the 5′ terminal and 3′ terminal of the RNA to form circular RNA;
combining the circular RNA with a primer hybridizable to a region of the circular RNA or a sequence complementary to the circular RNA, RNA-dependent DNA polymerase, and DNA-dependent DNA polymerase to form an aqueous component;
introducing the aqueous component into a microcompartment of a water-in-oil emulsion; and
amplifying DNA from the circular RNA.

2. The method of claim 1, wherein the RNA is mRNA, tRNA, rRNA, miRNA, or a combination thereof.

3. The method of claim 1, wherein the RNA is a transcriptome expressed from a cell or virus.

4. The method of claim 1, wherein the RNA ligase is a thermostable ATP-dependent ligase that catalyzes intramolecular ligation (i.e. circularization) of ssRNA templates having a 5′-phosphate and a 3′-hydroxyl group.

5. The method of claim 1, wherein the RNA-dependent DNA polymerase, the DNA dependent DNA polymerase, or both have strand displacement activity.

6. The method of claim 1, wherein the amplification is strand displacement amplification.

7. The method of claim 1, wherein the aqueous component further comprises a reagent for reverse transcription reaction or a reagent for DNA polymerization.

8. The method of claim 1, wherein water-in-oil emulsion comprises no more than one RNA molecule per microcompartment.

9. The method of claim 1, wherein introducing the aqueous component into the microcompartment comprises comprises mixing the aqueous component, an oily component, and a non-ionic surfactant to to provide a water-in-oil emulsion.

10. The method of claim 9, wherein the non-ionic surfactant is a non-ionic surfactant that has a hydrophilic-lipophilic balance (HLB) value of 4 or less.

11. The method of claim 1, wherein the amplifying is performed under suitable conditions for at least one of RNA-dependent DNA polymerization and DNA-dependent DNA polymerization.

12. The method of claim 1, wherein the amplifying is performed until DNA amplification in each microcompartment reaches a saturation state.

13. The method of claim 1, wherein the amplifying is performed under isothermal conditions.

14. The method of claim 1, wherein the amplifying is performed at a temperature of about 40° C. to about 50° C.

15. A method of estimating the amount of RNA in a sample, the method comprising:
amplifying DNA from RNA in the sample according to the method of claim 1; and
estimating the amount of RNA in the sample based on the amount of amplified DNA.

16. The method of claim 15, further comprising estimating a ratio of different kinds of RNA species in the sample based on a ratio of different kinds of amplified DNA species.

17. A composition for amplifying DNA from RNA in a sample comprising a water-in-oil emulsion comprising microcompartments, wherein one or more microcompartments comprises an aqueous solution comprising one or more circular RNA, a primer that is hybridizable to a region of the circular RNA or a region complementary to the circular RNA, RNA-dependent DNA polymerase, and DNA-dependent DNA polymerase.


SEQUENCE LISTING (9 sequences)

SEQ ID NO: 1 - DNA, 40 bp, Artificial Sequence
  Synthetic (T7_actin-100mer-F)
gaaattaatacgactcactatacctggcctcgctgtccac

SEQ ID NO: 2 - DNA, 20 bp, Artificial Sequence
  Synthetic (T7_actin-100mer-R)
gtcatagtccgcctagaagc

SEQ ID NO: 3 - DNA, 40 bp, Artificial Sequence
  Synthetic (T7_GUSB-100mer-F)
gaaattaatacgactcactataccaggtatccccactcag

SEQ ID NO: 4 - DNA, 18 bp, Artificial Sequence
  Synthetic (T7_GUSB-100mer-R)
cgccctgactcggggagg

SEQ ID NO: 5 - DNA, 41 bp, Artificial Sequence
  Synthetic (T7_TFRC-100mer-F)
gaaattaatacgactcactatacctggactatgagaggtac

SEQ ID NO: 6 - DNA, 21 bp, Artificial Sequence
  Synthetic (T7_TFRC-100mer-R)
cagccactgtaaactcaggcc

SEQ ID NO: 7 - DNA, 23 bp, Artificial Sequence
  Synthetic (Actin TaqMan probe FAM is attached to 5′ end and BHQ1 is attached to 3′ end)
aggagtatgacgagtccggcccc

SEQ ID NO: 8 - DNA, 27 bp, Artificial Sequence
  Synthetic (GUSB  TaqMan probe FAM is attached to 5′ end and BHQ1 is attached to 3′ end)
tcaagtaaacgggctgttttccaaaca

SEQ ID NO: 9 - DNA, 28 bp, Artificial Sequence
  Synthetic (TFRC TaqMan probe FAM is attached to 5′ end and BHQ1 is attached to 3′ end)
agggatctgaaccaatacagagcagaca